(19) United States
(12) Patent Application Publication
(10) Pub. No.: US 2026/0035350 A1
(43) Pub. Date: Feb. 5, 2026

(54) METHOD FOR SYNTHESIZING SULFAMONOMETHOXINE

(71) Applicant: Nanhai Beisha Pharmaceutical Co., Ltd., Foshan (CN)

(72) Inventors: Weijie QIU, Foshan (CN); Laiyue LIU, Foshan (CN); Weifeng ZHUANG, Foshan (CN); Sheng HUANG, Foshan (CN); Junwen MO, Foshan (CN); Guangrui HUANG, Foshan (CN)

(21) Appl. No.: 18/989,206

(22) Filed: Dec. 20, 2024

(30) Foreign Application Priority Data
Jul. 31, 2024 (CN) 202411035490.9

(51) Int. Cl. C07D 239/47

(52) U.S. Cl. CPC C07D 239/47

(57) ABSTRACT

Disclosed is a method for synthesizing sulfamonomethoxine, relating to the technical field of drug preparation. 4-chloro-6-methoxypyrimidine, an alkali metal salt of sulfanilamide, and an organic solvent are mixed, and a resulting mixture is subjected to condensation reaction to obtain the sulfamonomethoxine.

### CROSS REFERENCE TO RELATED APPLICATION

This patent application claims the benefit and priority of Chinese Patent Application No. 2024110354909, entitled “METHOD FOR SYNTHESIZING SULFAMONOMETHOXINE” filed on Jul. 31, 2024, the disclosure of which is incorporated by reference herein in its entirety as part of the present application.

### TECHNICAL FIELD

The present disclosure relates to the technical field of drug preparation, and in particular to a method for synthesizing sulfamonomethoxine.

### BACKGROUND

Sulfonamides are the earliest synthetic antibiotics generally in white or light yellow crystalline powders. Since their emergence and application in the 1930s, the sulfonamides have been widely valued and studied as the most valuable antibacterial drugs due to easy production and storage, desirable efficacy, convenient use, and low price. Sulfonamides have a history of over 80 years, and over 8,500 sulfonamides have been synthesized, where over 20 types including sulfadiazine, sulfamonomethoxine, sulfamerazine, and sulfamethazine are commonly used in clinical practice. With the continuous discovery and development of various antibiotics, antibiotics and quinolones have gradually replaced the sulfonamides. However, the sulfonamides still exhibit their unique advantages such as wide antibacterial spectrum, stable properties, easy application, low price, and no food consumption in drug production, and ability of production in large quantities. The discovery of antibacterial enhancers such as trimethoprim (TMP) and diaveridine (DVD) has expanded the antibacterial spectrum and greatly enhanced the antibacterial activity of sulfonamides when being used in combination with the antibacterial enhancers. Therefore, sulfonamides are still one of the important drugs in the anti-infection treatment of livestock and poultry.

Sulfamonomethoxine, a type of the sulfonamides, is a white powdery solid composed of aniline and methoxylated pyrimidine linked by a sulfanilamide group. As a broad-spectrum antibacterial drug, the sulfamonomethoxine shows low toxicity and easy oral absorption, and has an antibacterial effect on various Gram-positive bacteria and some Gram-negative bacteria.

At present, the synthesis of sulfamonomethoxine involves mainly a two-step process. Sulfachloropyrimidine is prepared by condensation of 4,6-dichloropyrimidine with sodium sulfanilamide in N,N-dimethylformamide (DMF) for 4 h to 6 h; the sulfachloropyrimidine is purified and then reacted with sodium methoxide to obtain the sulfamonomethoxine. However, since the 4,6-dichloropyrimidine is unstable during the condensation reaction and generated sulfachloropyrimidine sodium is also easily degraded under same conditions, a yield of the product is low (75% to 80%).

### SUMMARY

In view of this, an object of the present disclosure is to provide a method for synthesizing sulfamonomethoxine. In the present disclosure, the synthesis method achieves a high yield of the sulfamonomethoxine.

To achieve the above object, the present disclosure provides the following technical solutions:

The present disclosure provides a method for synthesizing sulfamonomethoxine, including:
mixing 4-chloro-6-methoxypyrimidine, an alkali metal salt of sulfanilamide, and an organic solvent, and subjecting a resulting mixture to condensation reaction, to obtain the sulfamonomethoxine.

In some embodiments, a molar ratio of the 4-chloro-6-methoxypyrimidine to the alkali metal salt of sulfanilamide is in a range of 1:(2.05-2.15); and the alkali metal salt of sulfanilamide includes one selected from the group consisting of sodium sulfanilamide and potassium sulfanilamide.

In some embodiments, the organic solvent includes at least one selected from the group consisting of N,N-dimethylformamide (DMF) and N,N-dimethylacetamide (DMA).

In some embodiments, the condensation reaction is conducted at a temperature of 90° C. to 100° C. for 3.5 h to 4 h.

In some embodiments, the alkali metal salt of sulfanilamide is synthesized by a process including: mixing an alkali metal hydroxide, sulfanilamide, and water, and subjecting a resulting mixture to salification reaction, to obtain the alkali metal salt of sulfanilamide.

In some embodiments, the method further includes post-treatment after the condensation reaction, where the post-treatment includes:
concentrating a condensation reaction solution obtained by the condensation reaction to obtain a concentrated solution and a recovered organic solvent; where the recovered organic solvent is reused in the condensation reaction;
mixing the concentrated solution, a first water, and a first activated carbon, and subjecting a resulting mixture to a first decolorization and then a first solid-liquid separation to obtain a first liquid component; cooling the first liquid component to room temperature, adjusting a pH value of the first liquid component to 7.5-8, and subjecting a resulting system to a first crystallization and then a second solid-liquid separation to obtain a second liquid component and a second solid component; and drying the second solid component to obtain recovered sulfanilamide; where the recovered sulfanilamide is reused in preparing the alkali metal salt of sulfanilamide;
cooling the second liquid component to room temperature, adjusting a pH value of the second liquid component to 5-6, and subjecting a resulting mixture system to a second crystallization and then a third solid-liquid separation, to obtain a third solid component, namely a crude sulfamonomethoxine product;
subjecting the crude sulfamonomethoxine product, a second water, and an alkaline reagent to heat dissolution, adding a second activated carbon thereto, and subjecting an obtained mixture to a second decolorization and then thermal solid-liquid separation, to obtain a hot liquid component (i.e., a third liquid component); and
adjusting a pH value of the hot liquid component to 5-6, subjecting an obtained system to cooling crystallization and then a fourth solid-liquid separation, to obtain a fourth solid component, and drying the fourth solid component, to obtain a refined sulfamonomethoxine product.

In some embodiments, a mass ratio of the alkali metal salt of sulfanilamide to the first activated carbon is in a range of 100:(1-2);
a mass ratio of the alkali metal salt of sulfanilamide to the first water is in a range of 1:(1.4-1.6); and
the first decolorization is conducted at a temperature of 90° C. to 95° C. for 1.5 h to 2 h.

In some embodiments, the first crystallization and the second crystallization each are conducted at a temperature of 20° C. to 25° C. for 1.5 h to 2 h.

In some embodiments, a mass ratio of the crude sulfamonomethoxine product to the second water is in a range of 1:(4.75-5.25);
a molar ratio of the crude sulfamonomethoxine product to the alkaline agent is in a range of 1:(0.5-0.51), and the alkaline reagent includes at least one selected from the group consisting of calcium hydroxide, sodium hydroxide, and potassium hydroxide;
a mass ratio of the crude sulfamonomethoxine product to the second activated carbon is in a range of 100:(3-5); and
the heat dissolution, the second decolorization, and the thermal solid-liquid separation each are independently conducted at a temperature of 90° C. to 95° C., and the second decolorization is conducted for 1.5 h to 2 h.

In some embodiments, the cooling crystallization is conducted at a temperature of 5° C. to 15° C. for 1.5 h to 2 h.

4-chloro-6-methoxypyrimidine, an alkali metal salt of sulfanilamide, and an organic solvent are mixed, and a resulting mixture is subjected to condensation reaction, to obtain the sulfamonomethoxine. The synthesis method has the advantages of a high product yield and product purity; desirable quality; cheap and readily available raw materials; simple, safe, and stable preparation process with high efficiency and environmental friendliness; mild reaction conditions; simple operations; and convenience for industrial production. As shown in test results of the examples, the sulfamonomethoxine prepared by the method has a high yield of not less than 91.5%.

The alkali metal salt of sulfanilamide is prepared by subjecting an alkali metal hydroxide, sulfanilamide, and water to salification reaction, and there is no need to purify and separate the alkali metal salt of sulfanilamide. Therefore, the post-treatment is simple, thereby reducing production cost of the sulfamonomethoxine and generating less waste liquid.

The sulfamonomethoxine obtained by the post-treatment has a high purity, the post-treatment has simple steps, and the organic solvent and the sulfanilamide recovered in the post-treatment could be reused, thereby reducing the production cost of the sulfamonomethoxine and greatly reducing sewage generation.

### BRIEF DESCRIPTION OF THE DRAWINGS

FIG. 1 shows the infrared spectrum of sulfamonomethoxine prepared in Example 1.

FIG. 2 shows the infrared spectrum of a standard sample of sulfamonomethoxine.

### DETAILED DESCRIPTION OF THE EMBODIMENTS

The present disclosure provides a method for synthesizing sulfamonomethoxine, including:
mixing 4-chloro-6-methoxypyrimidine, an alkali metal salt of sulfanilamide, and an organic solvent, and subjecting a resulting mixture to condensation reaction, to obtain the sulfamonomethoxine.

In the present disclosure, unless otherwise specified, all materials and equipment used are commercially available items in the art.

In some embodiments of the present disclosure, a molar ratio of the 4-chloro-6-methoxypyrimidine to the alkali metal salt of sulfanilamide is in a range of 1:(2.05-2.15), and preferably 1:2.1. In some embodiments, the alkali metal salt of sulfanilamide includes sodium sulfanilamide or potassium sulfanilamide, and preferably is sodium sulfanilamide.

In some embodiments of the present disclosure, the alkali metal salt of sulfanilamide is synthesized by a process including: mixing an alkali metal hydroxide, sulfanilamide, and water, and subjecting a resulting mixture to salification reaction, to obtain the alkali metal salt of sulfanilamide. In some embodiments, the alkali metal hydroxide includes sodium hydroxide or potassium hydroxide. In some embodiments, a mass ratio of the sulfanilamide to the alkali metal hydroxide is in a range of 1:(0.227-0.232), and preferably 1:0.023. In some embodiments, a mass ratio of the sulfanilamide to the water is in a range of 1:(2.75-3.25), and preferably 1:(2.8-3). In some embodiments of the present disclosure, the salification reaction is conducted at a temperature of 90° C. to 100° C., and preferably 93° C. to 95° C. In some embodiments, the salification reaction is conducted for 20 min to 40 min, and preferably 30 min. In some embodiments, the salification reaction is conducted under stirring.

In some embodiments of the present disclosure, the method further includes: after the salification reaction, subjecting a salification reaction solution obtained by the salification reaction to evaporation to dryness, to obtain the alkali metal salt of sulfanilamide. In some embodiments, the evaporation to dryness is conducted by reduced-pressure distillation until the water is completely removed.

In some embodiments of the present disclosure, the organic solvent includes DMF and/or DMA. In some embodiments, the organic solvent is an anhydrous organic solvent. In some embodiments, a solid-to-liquid ratio of the 4-chloro-6-methoxypyrimidine to the organic solvent is in a range of 1 g:(3-4) mL, and preferably is 1 g:3.5 mL.

In some embodiments of the present disclosure, the condensation reaction is conducted at a temperature of 90° C. to 100° C., and preferably 95° C. In some embodiments, the condensation reaction is conducted for 3.5 h to 4 h. In some embodiments, the condensation reaction is conducted under stirring.

In some embodiments of the present disclosure, the method further includes post-treatment after the condensation reaction, where the post-treatment includes:
concentrating a condensation reaction solution obtained by the condensation reaction to obtain a concentrated solution and a recovered organic solvent; where the recovered organic solvent is reused in the condensation reaction;
mixing the concentrated solution, a first water, and a first activated carbon, and subjecting a resulting mixture to a first decolorization and then a first solid-liquid separation to obtain a first liquid component; cooling the first liquid component to room temperature, adjusting a pH value of the first liquid component to 7.5-8, and subjecting a resulting system to a first crystallization and then a second solid-liquid separation to obtain a second liquid component and a second solid component; and drying the second solid component to obtain recovered sulfanilamide;
cooling the second liquid component to room temperature, adjusting a pH value of the second liquid component to 5-6, and subjecting a resulting system to a second crystallization and then a third solid-liquid separation, to obtain a third solid component, namely a crude sulfamonomethoxine product;
subjecting the crude sulfamonomethoxine product, a second water, and an alkaline reagent to heat dissolution, adding a second activated carbon thereto, and subjecting a resulting mixture to a second decolorization and then thermal solid-liquid separation, to obtain a hot liquid component (i.e., a third liquid component); and
adjusting a pH value of the hot liquid component to 5-6, and subjecting a resulting system to cooling crystallization and then a fourth solid-liquid separation to obtain a fourth solid component, and drying the fourth solid component, to obtain a refined sulfamonomethoxine product.

In some embodiments of the present disclosure, a condensation reaction solution obtained by the condensation reaction is concentrated to obtain a concentrated solution and a recovered organic solvent; where the recovered organic solvent is reused in the condensation reaction. In some embodiments, the concentration is performed by reduced-pressure concentration. In the present disclosure, there is no particular limitation on conditions for the concentration, as long as the organic solvent could be completely recovered.

In some embodiments of the present disclosure, the concentrated solution, the first water, and the first activated carbon are mixed, and a resulting mixture is subjected to a first decolorization and then a first solid-liquid separation to obtain a first liquid component; the first liquid component is cooled to room temperature (20° C. to 25° C.), a pH value of the first liquid component is adjusted to 7.5-8, and the first liquid component is subjected to crystallization and then a second solid-liquid separation to obtain a second liquid component and a solid component; and the solid component is dried to obtain recovered sulfanilamide. In some embodiments, a mass ratio of the alkali metal salt of sulfanilamide to the first activated carbon is in a range of 100:(1-2), and preferably is 100:1.5. In some embodiments, a mass ratio of the alkali metal salt of sulfanilamide to the first water is in a range of 1:(1.4-1.6), and preferably is 1:1.5. In some embodiments, the first water is hot water, and the hot water is preferably at a temperature of 85° C. to 100° C., more preferably 90° C. to 95° C. In some embodiments, the first decolorization is conducted at a temperature of 90° C. to 95° C., and preferably at 93° C. to 95° C. In some embodiments, the first decolorization is conducted for 1.5 h to 2 h, and preferably 2 h. In some embodiments, the first solid-liquid separation and the second solid-liquid separation each independently include filtration, suction filtration, or centrifugation. In some embodiments, an acid for adjusting the pH value of the first liquid component to 7.5-8 includes at least one of acetic acid, hydrochloric acid, sulfuric acid, and phosphoric acid. In some embodiments, the pH value is adjusted to 7.6-7.9, and preferably 7.7-7.8. In some embodiments, the drying is conducted at a temperature of 95° C. to 105° C., and preferably 100° C. In the present disclosure, there is no special limitation to a drying time, as long as a constant weight could be achieved.

In some embodiments of the present disclosure, the second liquid component is cooled to room temperature (20° C. to 25° C.), a pH value of the second liquid component is adjusted to 5-6, and the second liquid component is subjected to crystallization and then a third solid-liquid separation, to obtain a solid component, namely a crude sulfamonomethoxine product. In some embodiments, an acid for adjusting the pH value of the second liquid component to 5-6 includes at least one of acetic acid, hydrochloric acid, sulfuric acid, and phosphoric acid. In some embodiments, the pH value is adjusted to 5.2-5.8, and preferably 5.4-5.6. In some embodiments, the crystallization is conducted at a temperature of 20° C. to 25° C. (room temperature). In some embodiments, the crystallization is conducted for 1.5 h to 2 h. In some embodiments, the third solid-liquid separation independently includes filtration, suction filtration, or centrifugation.

In some embodiments of the present disclosure, after obtaining the crude sulfamonomethoxine product, the crude sulfamonomethoxine product, a second water, and an alkaline reagent are subjected to heat dissolution, a second activated carbon is added thereto, and a resulting mixture is subjected to a second decolorization, and then thermal solid-liquid separation, to obtain a hot liquid component. In some embodiments, a mass ratio of the crude sulfamonomethoxine product to the second water is in a range of 1:(4.75-5.25), and preferably 1:(4.8-5). In some embodiments, a molar ratio of the crude sulfamonomethoxine product to the alkaline reagent is in a range of 1:(0.5-0.51), and preferably 1:0.55. In some embodiments, the alkaline reagent includes at least one of calcium hydroxide, sodium hydroxide, and potassium hydroxide, and preferably is calcium hydroxide. In some embodiments, a mass ratio of the crude sulfamonomethoxine product to the second activated carbon is in a range of 100:(3-5), and preferably 100:4. In some embodiments, the heat dissolution, the second decolorization, and the thermal solid-liquid separation each are independently conducted at a temperature of 90° C. to 95° C., and preferably 92° C. to 93° C. In some embodiments, the second decolorization is conducted for 1.5 h to 2 h, and preferably 2 h. In some embodiments, the thermal solid-liquid separation independently includes thermal filtration, thermal suction filtration, or thermal centrifugation.

In some embodiments of the present disclosure, after obtaining the hot liquid component, a pH value of the hot liquid component is adjusted to 5-6, and the resulting system is subjected to cooling crystallization and then a fourth solid-liquid separation, and an obtained solid component is dried to obtain a refined sulfamonomethoxine product. In some embodiments, an acid for adjusting the pH value of the hot liquid component to 5-6 includes at least one of acetic acid, hydrochloric acid, sulfuric acid, and phosphoric acid. In some embodiments, the pH value is adjusted to 5.2-5.8, and preferably 5.4-5.6. In some embodiments, the cooling crystallization is conducted at a temperature of 5° C. to 15° C., preferably 10° C. In some embodiments, the cooling crystallization is conducted for 1.5 h to 2 h.

To further illustrate the present disclosure, the method for synthesizing sulfamonomethoxine according to the present disclosure is described in detail below in conjunction with examples, but these examples should not be construed as limiting the claimed scope of the present disclosure.

### Example 1

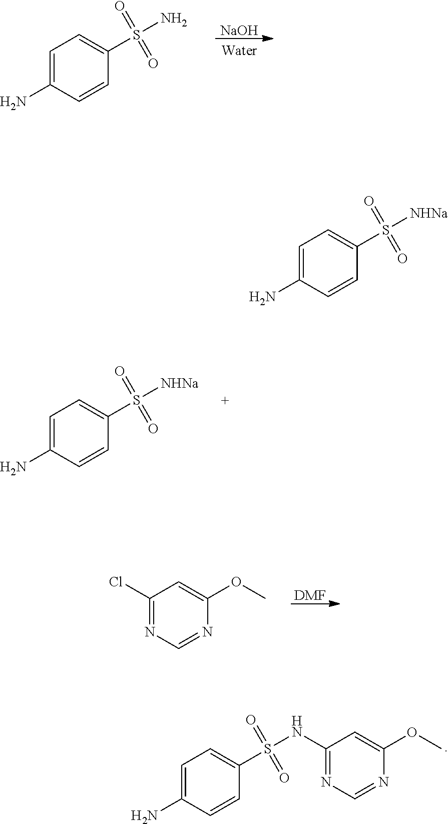

950 g of purified water was added into a reaction container, 78.5 g of sodium hydroxide was added thereto at 30° C., and 345 g of sulfanilamide was then added thereto. A resulting mixture was heated to 95° C. with stirring and reacted for 0.5 h, and the water therein was evaporated to obtain sodium sulfanilamide.

350 mL of DMF, 100 g of 4-chloro-6-methoxypyrimidine, and 276.5 g of sodium sulfanilamide were added into the reaction container, and a resulting mixture was heated to 100° C. with stirring and reacted for 4 h. The DMF was recovered under reduced pressure (the DMF was reused for the next batch of condensation). 390 mL of 95° C. hot water and 5 g of activated carbon were added thereto, and a resulting mixture was subjected to decolorization at 95° C. for 2 h. A resulting system was subjected to thermal filtration, and a filtrate was cooled to 25° C., and adjusted to pH=7.5 with acetic acid. A solid was then precipitated, and a resulting system was filtered to obtain a filtrate and a filter cake. The filter cake was dried to obtain recovered sulfanilamide (the recovered sulfanilamide was reused for the preparation of the next batch of sodium sulfanilamide).

The filtrate was adjusted to pH=5.0 with acetic acid at a temperature of 20° C. to 25° C., and a resulting system was subjected to crystallization for 2 h. A resulting mixture was filtered, and a filter cake obtained was dried to obtain a crude sulfamonomethoxine product (182.7 g, a crude product molar yield of 94.3%).

100 g of the crude sulfamonomethoxine product, 500 g of purified water, and 13.3 g of calcium hydroxide were added into the reaction container, and a resulting mixture was heated to 95° C. for dissolution. 5 g of activated carbon was added thereto, and a resulting mixture was subjected to decolorization at 95° C. for 2 h. A resulting system was subjected to thermal filtration, a filtrate was adjusted with acetic acid to pH=5.0 at 95° C., then cooled to 5° C., and subjected to crystallization for 2 h. A resulting mixture was filtered, and a filter cake obtained was dried to obtain a refined sulfamonomethoxine product (97.2 g, with a purity of 99.61%, and a refined yield of 97.2%).

### Example 2

1,120 g of purified water was added into a reaction container, 80 g of sodium hydroxide was added thereto at 30° C., and 345 g of sulfanilamide was then added thereto. A resulting mixture was heated to 95° C. with stirring and reacted for 0.5 h, and the water therein was evaporated to obtain sodium sulfanilamide.

350 mL of DMF, 100 g of 4-chloro-6-methoxypyrimidine, and 290 g of sodium sulfanilamide were added into the reaction container, and a resulting mixture was heated to 100° C. with stirring and reacted for 3.5 h. The DMF was recovered under reduced pressure (the DMF was reused for the next batch of condensation). 460 mL of 95° C. hot water and 5 g of activated carbon were added thereto, and a resulting mixture was subjected to decolorization at a temperature of 90° C. to 95° C. for 2 h. A resulting system was subjected to thermal filtration, and a filtrate was cooled to 25° C., and adjusted with acetic acid to pH=8.0. A solid was then precipitated, and a resulting system was filtered to obtain a filtrate and a filter cake. The filter cake was dried to obtain recovered sulfanilamide (the recovered sulfanilamide was reused for the preparation of the next batch of sodium sulfanilamide).

The filtrate was adjusted with acetic acid to pH=6.0 at a temperature of 20° C. to 25° C., and a resulting system was subjected to crystallization for 2 h. A resulting mixture was filtered, and a filter cake obtained was dried to obtain a crude sulfamonomethoxine product (184.7 g, a crude product molar yield of 95.0%).

100 g of the crude sulfamonomethoxine product, 500 g of purified water, and 13.5 g of calcium hydroxide were added into the reaction container, and a resulting mixture was heated to 95° C. for dissolution. 5 g of activated carbon was added thereto, and a resulting mixture was subjected to decolorization at 95° C. for 2 h. A resulting system was subjected to thermal filtration, and a filtrate was adjusted with acetic acid to pH=6.0 at 95° C., then cooled to 15° C., and subjected to crystallization for 2 h. A resulting mixture was filtered, and a filter cake obtained was dried to obtain a refined sulfamonomethoxine product (98.3 g, with a purity of 99.59%, and a refined yield of 98.3%).

### Example 3

1,120 g of purified water was added into a reaction container, 110 g of potassium hydroxide was added thereto at 15° C., and 345 g of sulfanilamide was then added thereto. A resulting mixture was heated to 95° C. with stirring and reacted for 0.5 h, and the water therein was evaporated to obtain potassium sulfanilamide.

350 mL of DMF, 100 g of 4-chloro-6-methoxypyrimidine, and 299 g of potassium sulfanilamide were added into the reaction container, and a resulting mixture was heated to 100° C. with stirring and reacted for 4 h. The DMF therein was recovered under reduced pressure (the DMF was reused for the next batch of condensation). 400 mL of 95° C. hot water and 6 g of activated carbon were added thereto, and a resulting mixture was subjected to decolorization at 95° C. for 2 h. A resulting system was subjected to thermal filtration, and a filtrate was cooled to 25° C., and adjusted with acetic acid to pH=7.8. A solid was then precipitated, and a resulting system was filtered to obtain a filtrate and a filter cake. The filter cake was dried to obtain recovered sulfanilamide (the recovered sulfanilamide was reused for the preparation of the next batch of potassium sulfanilamide or sodium sulfanilamide).

The filtrate was adjusted with acetic acid to pH=5.5 at a temperature of 20° C. to 25° C., and a resulting system was subjected to crystallization for 2 h. A resulting mixture was filtered, and a filter cake obtained was dried to obtain a crude sulfamonomethoxine product (184.3 g, a crude product molar yield of 94.8%).

100 g of the crude sulfamonomethoxine product, 500 g of purified water, and 13.3 g of calcium hydroxide were added into the reaction container, and a resulting mixture was heated to 95° C. for dissolution. 5 g of activated carbon was added thereto, and a resulting mixture was subjected to decolorization at 95° C. for 2 h. A resulting system was subjected to thermal filtration, and a filtrate obtained was adjusted with acetic acid to pH=5.5 at 95° C., then cooled to 10° C., and subjected to crystallization for 2 h. A resulting mixture was filtered, and a filter cake obtained was dried to obtain a refined sulfamonomethoxine product (97.8 g, with a purity of 99.69%, and a refined yield of 97.8%).

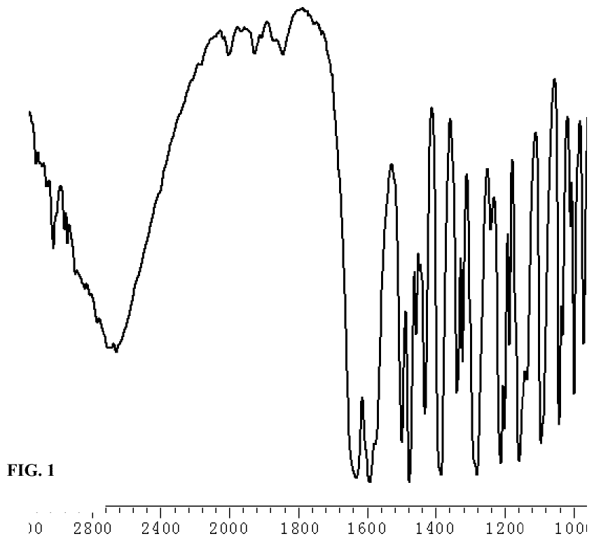

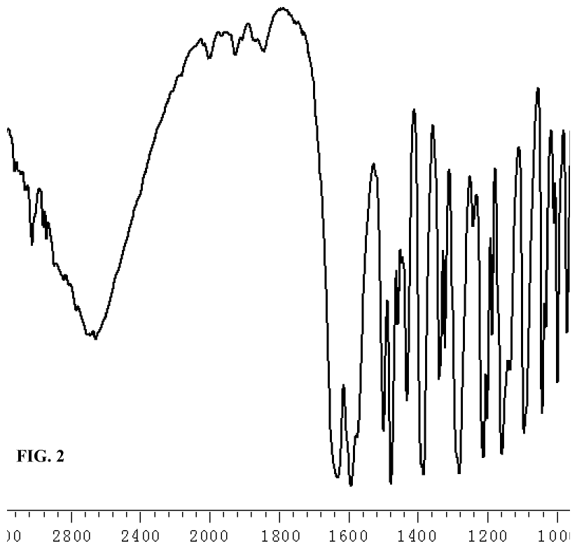

FIG. 1 shows the infrared spectrum of sulfamonomethoxine prepared in Example 1; and FIG. 2 shows the infrared spectrum of a standard sample of sulfamonomethoxine (purchased from the China Institute of Veterinary Drug Control, IVDC). Comparison of the results in FIG. 1 and FIG. 2 shows that the infrared spectra of the two are consistent, indicating that the synthetic method according to the present disclosure could prepare sulfamonomethoxine successfully.

The above descriptions are merely preferred embodiments of the present disclosure. It should be noted that a person of ordinary skill in the art may further make several improvements and modifications without departing from the principle of the present disclosure, but such improvements and modifications should be deemed as falling within the scope of the present disclosure.

What is claimed is:

1. A method for synthesizing sulfamonomethoxine, comprising:
mixing 4-chloro-6-methoxypyrimidine, an alkali metal salt of sulfanilamide, and an organic solvent; and
subjecting a resulting mixture to condensation reaction, to obtain the sulfamonomethoxine.

2. The method as claimed in claim 1, wherein a molar ratio of the 4-chloro-6-methoxypyrimidine to the alkali metal salt of sulfanilamide ranges from 1:2.05 to 1:2.15; and
the alkali metal salt of sulfanilamide comprises one selected from the group consisting of sodium sulfanilamide and potassium sulfanilamide.

3. The method as claimed in claim 1, wherein the organic solvent comprises at least one selected from the group consisting of N,N-dimethylformamide and N,N-dimethylacetamide.

4. The method as claimed in claim 1, wherein the condensation reaction is conducted at a temperature of 90° C. to 100° C. for 3.5 h to 4 h.

5. The method as claimed in claim 2, wherein the condensation reaction is conducted at a temperature of 90° C. to 100° C. for 3.5 h to 4 h.

6. The method as claimed in claim 3, wherein the condensation reaction is conducted at a temperature of 90° C. to 100° C. for 3.5 h to 4 h.

7. The method as claimed in claim 1, wherein the alkali metal salt of sulfanilamide is synthesized by a process comprising: mixing an alkali metal hydroxide, sulfanilamide, and water, and subjecting a resulting mixture to salification reaction, to obtain the alkali metal salt of sulfanilamide.

8. The method as claimed in claim 2, wherein the alkali metal salt of sulfanilamide is synthesized by a process comprising: mixing an alkali metal hydroxide, sulfanilamide, and water, and subjecting a resulting mixture to salification reaction, to obtain the alkali metal salt of sulfanilamide.

9. The method as claimed in claim 4, further comprising post-treatment after the condensation reaction, wherein the post-treatment comprises:
concentrating a condensation reaction solution obtained by the condensation reaction to obtain a concentrated solution and a recovered organic solvent; wherein the recovered organic solvent is reused in the condensation reaction;
mixing the concentrated solution, a first water, and a first activated carbon, and subjecting a resulting mixture to a first decolorization and then a first solid-liquid separation to obtain a first liquid component; cooling the first liquid component to room temperature, adjusting a pH value of the first liquid component to 7.5-8, and subjecting a resulting system to a first crystallization and then a second solid-liquid separation to obtain a second liquid component and a second solid component; and drying the second solid component to obtain recovered sulfanilamide; wherein the recovered sulfanilamide is reused in preparing the alkali metal salt of sulfanilamide;
cooling the second liquid component to room temperature, adjusting a pH value of the second liquid component to 5-6, and subjecting a resulting mixture system to a second crystallization and then a third solid-liquid separation, to obtain a third solid component, namely a crude sulfamonomethoxine product;
subjecting the crude sulfamonomethoxine product, a second water, and an alkaline reagent to heat dissolution, adding a second activated carbon thereto, and subjecting an obtained mixture to a second decolorization and then thermal solid-liquid separation, to obtain a third liquid component; and
adjusting a pH value of the third liquid component to 5-6, subjecting an obtained system to cooling crystallization and then a fourth solid-liquid separation to obtain a fourth solid component, and drying the fourth solid component, to obtain a refined sulfamonomethoxine product.

10. The method as claimed in claim 9, wherein a mass ratio of the alkali metal salt of sulfanilamide to the first activated carbon ranges from 100:1 to 100:2;
a mass ratio of the alkali metal salt of sulfanilamide to the first water ranges from 1:1.4 to 1:1.6; and
the first decolorization is conducted at a temperature of 90° C. to 95° C. for 1.5 h to 2 h.

11. The method as claimed in claim 9, wherein the first crystallization and the second crystallization each are conducted at a temperature of 20° C. to 25° C. for 1.5 h to 2 h.

12. The method as claimed in claim 9, wherein a mass ratio of the crude sulfamonomethoxine product to the second water ranges from 1:4.75 to 1:5.25;
a molar ratio of the crude sulfamonomethoxine product to the alkaline agent ranges from 1:0.5 to 1:0.51, and the alkaline reagent comprises at least one selected from the group consisting of calcium hydroxide, sodium hydroxide, and potassium hydroxide;
a mass ratio of the crude sulfamonomethoxine product to the second activated carbon ranges from 100:3 to 100:5; and
the heat dissolution, the second decolorization, and the thermal solid-liquid separation each are independently conducted at a temperature of 90° C. to 95° C., and the second decolorization is conducted for 1.5 h to 2 h.

13. The method as claimed in claim 9, wherein the cooling crystallization is conducted at a temperature of 5° C. to 15° C. for 1.5 h to 2 h.
